(19) United States
(12) Patent Application Publication
(10) Pub. No.: US 2026/0345341 A1
(43) Pub. Date: Nov. 5, 2026

(54) CUSTOMIZABLE IMPLANT

(71) Applicant: NEXUS SPINE, LLC, Salt Lake CIty, UT (US)

(72) Inventors: David T. Hawkes, Salt Lake City, UT (US); Peter A. Halverson, Salt Lake City, UT (US)

(21) Appl. No.: 19/664,719

(22) Filed: Apr. 30, 2026

Related U.S. Application Data
(60) Provisional application No. 63/798,461, filed on May 1, 2025.
(60) Provisional application No. 63/914,999, filed on Nov. 10, 2025.

(51) Int. Cl. A61F 2/44

(52) U.S. Cl. CPC A61F 2/447

(57) ABSTRACT

Systems and methods for providing a multiple-footprint implant are provided. In some implementations, an interbody implant is provided. In some cases, the implant includes an implant body and one or more detachable segments selectively coupled to the implant body. According to some implementations, the implant has a first characteristic (e.g., size, footprint, angle, volume, shape, or other characteristic) when the detachable segment is coupled to the implant body, and a second characteristic (e.g., a different size, footprint, angle, volume, shape, or other characteristic) when the detachable segment is detached from the implant body. Other implementations are described.

### CROSS-REFERENCE TO RELATED APPLICATIONS

This application claims priority to United States Provisional Application No. 63/798,461, entitled “VARIABLE FOOTPRINT IMPLANT” (Attorney Docket No. 23845.228), and filed May 1, 2025, and to United States Provisional Application No. 63/914,999, entitled “CUSTOMIZABLE IMPLANT” (Attorney Docket No. 23845.239), and filed Nov. 10, 2025; the entire disclosures of each of which are incorporated herein by reference.

### FIELD

The present disclosure relates to the medical field, and more particularly to medical implants.

### BACKGROUND

Some traditional spinal implants are provided in a broad array of sizes to accommodate a range of patient anatomy, varying amounts of surgical access, varying surgical techniques, and different access methods. Thus, many implants come in multiple sizes, including different heights, footprints, lordotic angles, kyphotic angles, or other variations in size or shape. The instruments used to size, measure, and prepare the disc space for the implant are often made to match the implants. Because of this, some surgical centers often have relatively large inventories of implants and instruments on hand to ensure that the proper sizes and configurations are in stock during surgery. Additionally, it is common for a surgeon to discover intraoperatively that there is a need for a custom size that is not readily available. In some competing devices, it is possible that an incorrect implant is selected, which can result in the implant becoming contaminated and subsequently wasted. Thus, some traditional offerings can be cumbersome, expensive, and can present other challenges to medical professionals, to the surgery center, and to the manufacturer.

Thus, while techniques currently exist to provide medical implants, such as spinal implants, challenges still exist, including those listed above. It would be an improvement in the art to augment or even replace current techniques with other techniques.

### SUMMARY

Systems and methods for providing a multiple-footprint (or otherwise customizable) implant are provided. In some implementations, an interbody implant is provided. In some cases, the implant includes an implant body and one or more detachable segments that are selectively coupled to the implant body. According to some implementations, the implant has a first characteristic (e.g., footprint, contact surface area, angle, size, shape, volume, or any other suitable characteristic) when the detachable segment is coupled to the implant body, and a second characteristic when the detachable segment is detached from the implant body.

According to some implementations, an interbody implant is provided. In some cases, the interbody implant includes an implant body. In some cases, the interbody implant includes a first detachable segment selectively coupled to the implant body, wherein the interbody implant has a first characteristic (e.g., size) when the first detachable segment is coupled to the implant body, and a second characteristic when the first detachable segment is detached from the implant body.

In some implementations, the first detachable segment is coupled to a lateral portion of the implant body. In some implementations, the first detachable segment is coupled to at least one of a ventral portion and a dorsal portion of the implant body. In some implementations, the first detachable segment is coupled to at least one of a cranial portion and a caudal portion of the implant body.

In some cases, the interbody implant includes a second detachable segment, and in some cases, it includes a third detachable segment.

Where multiple detachable segments are included, this can be done in different ways. For example, in some cases, the second detachable segment is coupled to the first detachable segment. In some cases, the second detachable segment is disposed across the implant body from the first detachable segment, substantially opposite the first detachable segment. In some cases, the first detachable segment is coupled to at least one of a first lateral portion of the implant body, a second lateral portion of the implant body, a ventral portion of the implant body, a dorsal portion of the implant body, a cranial portion of the implant body, and a caudal portion of the implant body, wherein the second detachable segment is coupled to a different one of the first lateral portion of the implant body, the second lateral portion of the implant body, the ventral portion of the implant body, the dorsal portion of the implant body, the cranial portion of the implant body, and the caudal portion of the implant body.

According to some implementations, the first detachable segment is coupled to the implant body via a coupling comprising a breakaway portion. In some implementations, the breakaway portion is disposed within a recess in at least one of the implant body and the first detachable segment.

According to some implementations, the interbody implant includes a cranial surface and a caudal surface. In some cases, the implant body is disposed between the cranial surface and the caudal surface.

In some implementations, the detachable segment is selectively coupled to the implant body via a coupling, and detachment of the detachable segment from the implant body modifies a characteristic of the interbody implant. In some cases, the characteristic includes a size. In some cases, the characteristic includes an angle.

Some implementations include an interface whereby a tool can be used to separate the detachable segment from the implant body. Some implementations have a gap between the implant body and the detachable segment. In some cases, at least a portion of the coupling is disposed within a recess in at least one of the implant body and the detachable segment. Some implementations of the implant body have a lock configured to prevent the detachment of the detachable segment from the implant body when the lock is engaged.

The described implant body and the detachable segments can have any suitable characteristic that allows the implant to have a customizable footprint, shape, or other characteristic. Indeed, in some embodiments, the implant body and one or more detachable segments comprise one or more coil packs or other porous structures.

Some implementations include a method of modifying an interbody implant. In some cases, the method includes detaching a detachable segment of the interbody implant from an implant body of the interbody implant to thereby modify a characteristic of the interbody implant. The method can also include using any other component of an implant as discussed herein for any of its intended purposes, in any suitable manner.

The instant disclosure also presents additional components, embodiments, and methods for providing customizable implants with many advantages over some traditional implants, as discussed in more detail below.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures, in which:

FIG. 1A shows a top plan view of an implant with one or more detachable segments, in accordance with some embodiments of the disclosed systems and methods;

FIG. 1B shows a top perspective view of an implant with one or more detachable segments used in connection with a tool, according to some embodiments;

FIG. 1C shows a top perspective view of an implant with a detachable segment that has been detached, in accordance with some embodiments;

FIG. 2 shows an elevation view of an implant with one or more detachable segments, in accordance with some embodiments;

FIG. 3 shows a top plan view of an implant with one or more detachable segments, in accordance with some embodiments;

FIGS. 4A and 4B show close-up views of breakaway portions of an implant with a detachable segment, in accordance with some embodiments;

FIGS. 5A and 5B each show an elevation view of an implant with one or more detachable segments, in accordance with some embodiments;

FIG. 6 shows a top front perspective view of an implant with one or more detachable segments, in accordance with some embodiments; and

FIGS. 7-8B show various views of an implant with one or more detachable segments, according to some embodiments.

### DETAILED DESCRIPTION

A description of some embodiments of the described systems and methods will now be given with reference to the figures. It is expected that the present systems and methods may take many other forms and shapes. Hence, the following disclosure is intended to be illustrative and not limiting, and the scope of the disclosure should be determined by reference to the appended claims.

Some traditional spinal fusion interbody implants are provided in a variety of different sizes, configurations, or other characteristics. For example, some implants can have varying heights, footprints (e.g., planforms defined by a superior or inferior contact surface), angles (e.g., lordotic angles, kyphotic angles, or other angles), shapes (e.g., planform geometry or profile geometry), sizes (e.g., one or more linear dimensions, such as width, depth, or height), volumes (e.g., a three-dimensional space occupied by the implant), and other characteristics. However, these characteristics can vary independently of each other. For example, if a certain implant set (as a non-limiting example, an anterior lumbar interbody fusion (ALIF) set) contains 3 different footprints, 3 different lordotic angles, and 5 different heights, then 45 different implants can be needed in each set (assuming only 1 implant is desired for each unique combination of these distinct characteristics). As the options for each characteristic and the number of different characteristics increase, the number of implants needed to be kept in stock for a surgery can also increase, in some cases exponentially. However, if the number of options decreases, this can result in a huge reduction in the necessary number of implants needed on hand for a surgery and can result in a substantial corresponding cost savings. Indeed, taking the above example, if just one footprint size is eliminated, the number of implants needed can decrease from 45 to 30. This could reasonably be expected to result in about a 33% savings in carrying costs, and it could also make relevant procedures much easier for medical professionals.

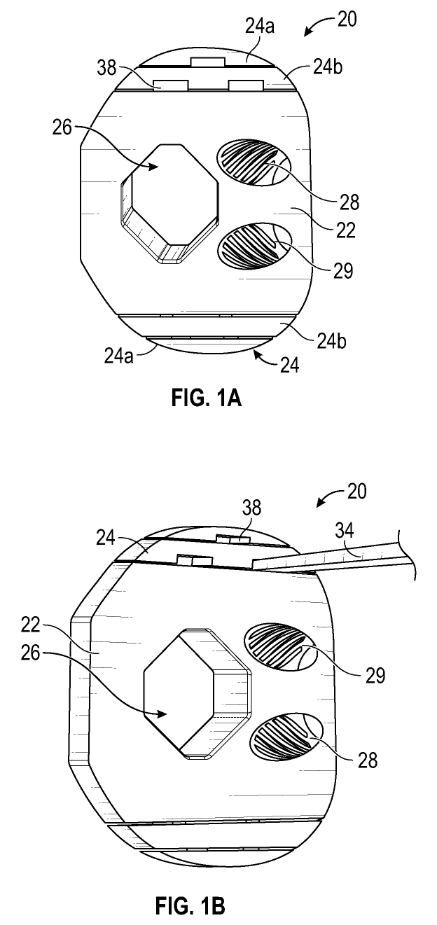

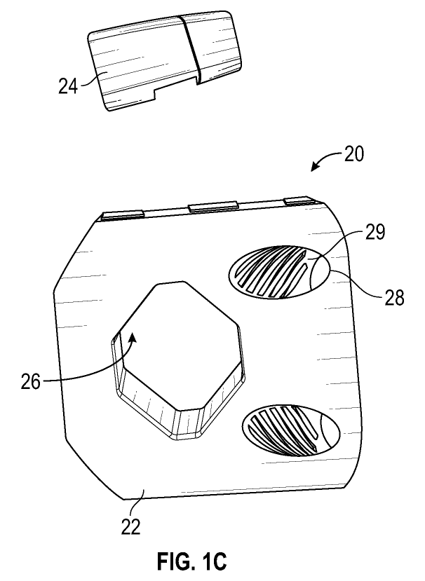

According to some embodiments of the instant systems and methods, an implant 20 that is selectively sizable or shapable to have multiple footprints, contact area sizes, angles, shapes, or other characteristics is provided. The implant can include any suitable type of spinal or other implant. For example, in some embodiments, the implant includes one or more of the following: interbody spacer implants (e.g., cervical spacers, anterior lumbar interbody fusion (ALIF) spacers, posterior lumbar interbody fusion (PLIF) spacers, transforaminal lumbar interbody fusion (TLIF) spacers, lateral lumbar interbody fusion (LLIF) spacers, oblique lumbar interbody fusion (OLIF) spacers, lordotic spacers, kyphotic spacers, expandable spacers, cylindrical spacers, box spacers, or any other kind of interbody spacers); hip implants (e.g., femoral stems, acetabular cups, or other hip implants); knee implants (e.g., tibial or femoral components, patellar buttons, or other knee implants); shoulder implants (e.g., glenoid components, humeral stems, or other shoulder implants); dental implants; plates (e.g., locking plates, compression plates, or other plates); anchors (e.g., nails, rods, screws, pins, wires, fixators, or other anchors); other spinal implants (e.g., pedicle screws, rods, or other spinal implant components); bone-anchored limb prostheses; cranial implants (e.g., mandibular reconstruction plates, orbital or cranial meshes or plates, or any other suitable cranial implants); or any other suitable implants. By way of non-limiting illustration, FIGS. 1A-6 show some embodiments of implants 20 substantially in the form of spinal interbody implants.

The implant 20 can include any suitable characteristics or components for operating as an interbody spacer implant (or other implant). For example, some embodiments of the implant include one or more: surfaces (e.g., cranial surfaces 21a, caudal surfaces 21b, ventral surfaces 21c, dorsal surfaces 21d, first lateral surfaces 21e, second lateral surfaces 21f, or other surfaces); implant bodies 22; detachable segments 24; passages 26; fixation mechanisms 28; porous structures; or any other suitable features.

The implant 20 can have any suitable characteristic that allows it to function as an implant (e.g., as an interbody implant). Indeed, in some embodiments, the implant body comprises one or more external or internal surfaces, such as one or more cranial (or superior) surfaces 21a that are configured to contact an endplate of a first (e.g., upper) vertebra, caudal (or inferior) surfaces 21b that are configured to contact an endplate of a second (e.g., lower) vertebra, ventral (or anterior) surfaces 21c, dorsal (or posterior) surfaces 21d, lateral (or left or right) surfaces 21e, 21f, or any other suitable surfaces.

In accordance with some embodiments, one or more contact surfaces of the implant 20 include one or more characteristics that allow for (or that promote) osseointegration. Indeed, in some embodiments one or more contact surfaces: define pores; have interconnected pores; have a roughened surface; comprise one or more materials to promote osteoconductivity or osteoinductivity, such as a hydroxyapatite or calcium phosphate coating configured to promote bone ingrowth; comprise a hydrophilic material; or have any other suitable characteristic.

Indeed, some embodiments of the implant 20 (e.g., the implant body 22, the detachable segments 24, or any other suitable component) include one or more porous structures. In such embodiments, the porous structure can include any suitable structures that are configured to facilitate bony in-growth into the implant body 22. For example, the porous structure can include any feature described in PCT application publication WO2017100366A1 (entitled “Porous Interbody Spacer”), the disclosure of which is incorporated herein by this reference in its entirety.

Indeed, in some embodiments, the porous structure of the implant body 22 or the detachable segments 24 (or any other suitable portion of the implant 20) includes any suitable structure having one or more gaps, spaces, air pockets, voids, orifices, cavities, hollows, open cells, closed cells, or any other pores capable of promoting bone growth into the implant. In some cases, the configuration of the porous structure at least partially gives rise to one or more of the load-bearing regions of the implant. Indeed, in some cases, the porous structure includes one or more coils, coil packs, strips, filaments, rods, fibers, sponge-like structures, lattice regions, scaffolds, honeycombs, mesh structures, woven structures, braided structures, nonwoven mats, reticulated structures, foam bodies, open-cell foams, closed-cell foams, porous matrices, trabecular structures, network structures, interconnecting struts, micro-architected frameworks, periodic cellular structures, stochastic cellular structures, gyroid structures, triply periodic minimal surface (TPMS) structures, perforated sheets, corrugated elements, folded structures, layered laminates, stacked elements, bundled fibers, entangled filaments, sintered bead structures, particulate agglomerates, re-entrant microstructures, hollow spheres, cages, capsules, tubular structures, channels, capillary networks, spring elements, helical elements, serpentine elements, zig-zag elements, compliant mechanisms, flexure-based structures, auxetic structures, anisotropic frameworks, graded-density structures, gradient porosity structures, composite porous bodies, and other strands configured to form spring-like, sponge-like, or other resilient or rigid structures that give rise to one or more desired stiffnesses of the load-bearing regions of the implant or detachable segments. Indeed, in some embodiments, the porous structure comprises one or more coils or coil packs.

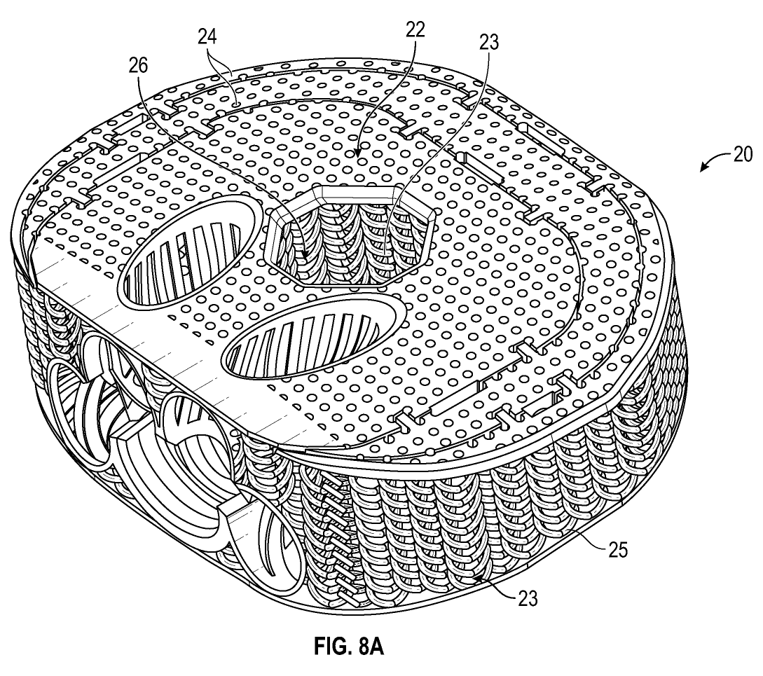

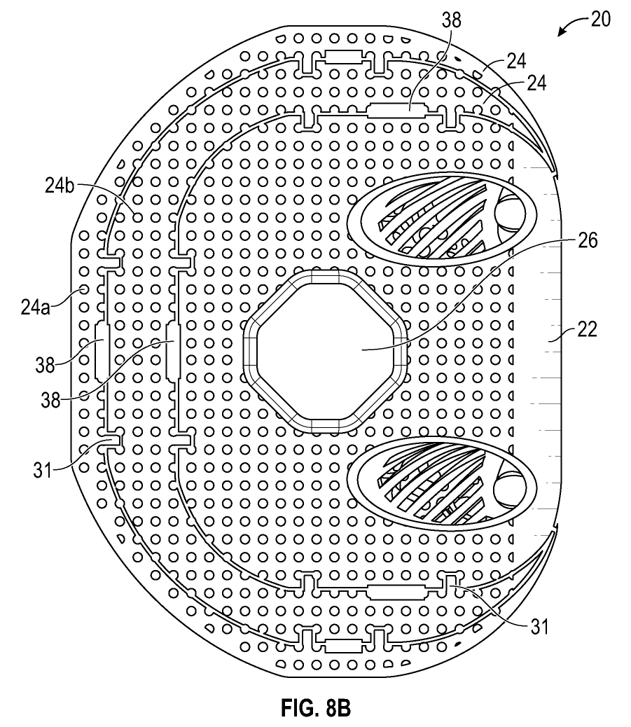

By way of non-limiting illustration, FIGS. 8A-8B show some embodiments in which at least one of the implant body 22 and the detachable segment 24 comprise a porous structure 23 (e.g., one or more coil packs 25). While such coil packs can (in accordance with some embodiments) vary throughout the implant 20 (e.g., within the implant body, between the implant body and a detachable segment, within a detachable segment, between different detachable segments, or in any other suitable manner), FIG. 8A shows some embodiments in which the porous structure 23 of the implant body 22 has a similar configuration to the porous structure 23 of the external-most detachable segment 24a.

According to some embodiments, the implant body 22 includes one or more passages 26. In this regard, the passage can include any suitable passage, including a passage configured to receive one or more anchors, additional structural components, or other materials. Indeed, in some embodiments, the passage is configured to receive a bone graft material, such as morselized bone prior to implantation of the implant, thereby providing a seed to better initiate bone growth into the implant 20 after implantation.

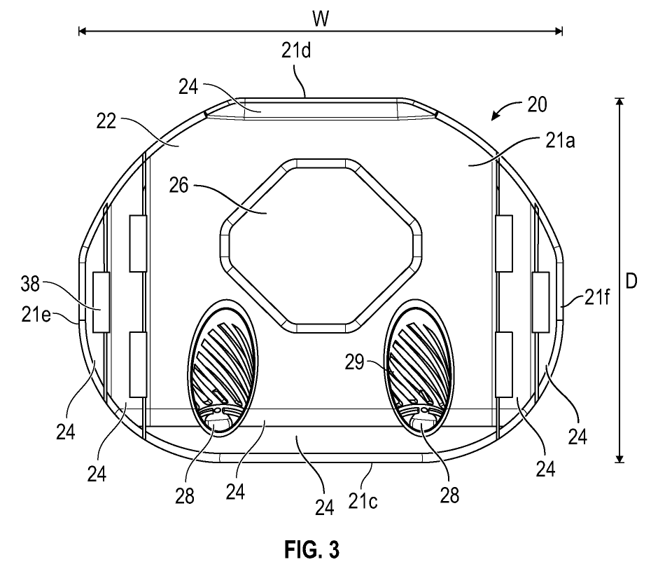

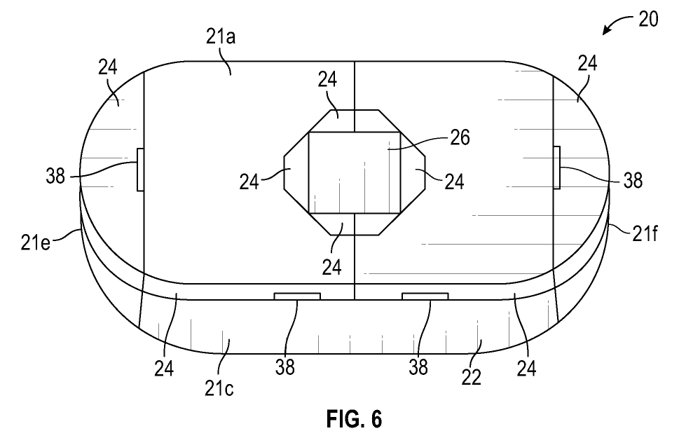

In some embodiments, the passage 26 is configured to have multiple sizes, shapes, or configurations, as desired on a patient-specific basis. In this regard, some embodiments of the detachable segments 24 (as discussed below) are included on an interior of the passage. Thus, such detachable segments can be removed to enlarge or change the shape of the graft window (or other passage). By way of non-limiting illustration, FIGS. 1A-1C and 3 show implants 20 having passages 26 configured to act as a graft window and receive a graft material therein. FIG. 6 shows an implant 20 having a passage 26 substantially in the form of a square, where the passage 26 is configured to be selectively enlarged or converted to an octagonal shape via removal of one or more detachable segments 24 bordering the passage 26.

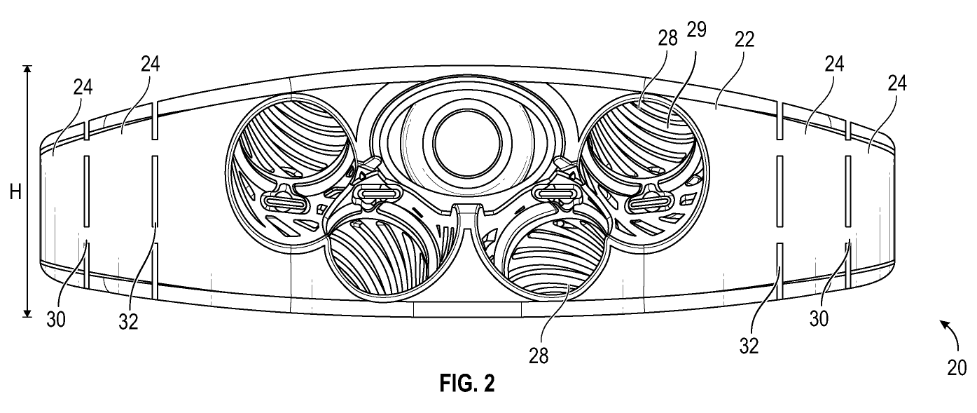

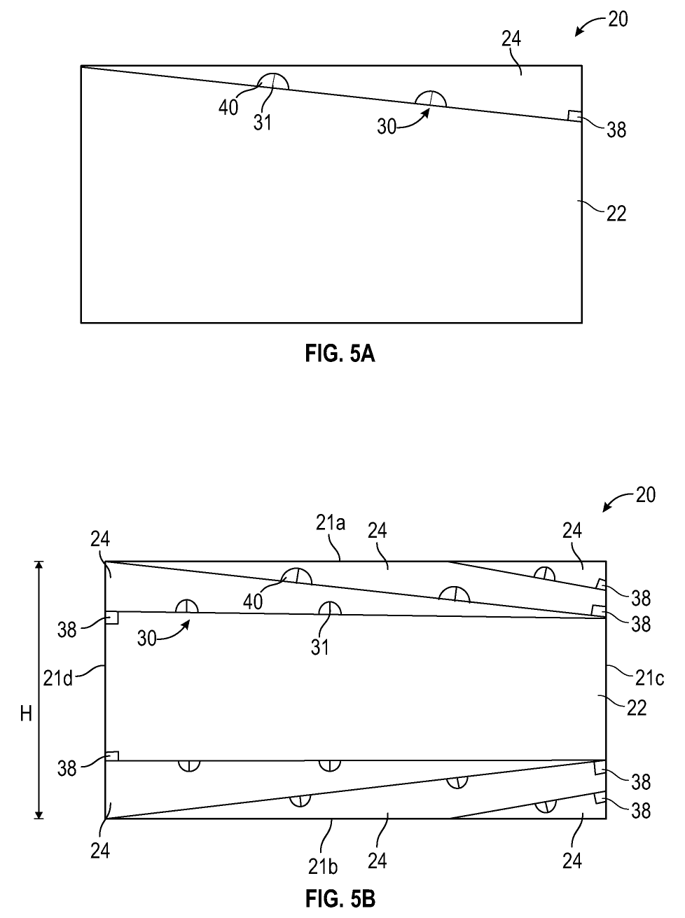

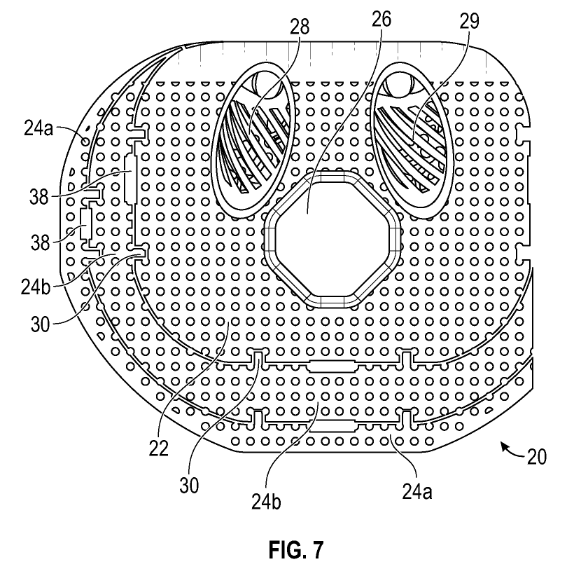

Regarding the detachable segments 24, the detachable segments can include any suitable component that is selectively detachable from the implant body 22 to thereby: provide a smaller footprint; provide a smaller contact area; change one or more angles of the implant 20; change the shape of the implant; modify or provide the passage 26; modify one or more fixation mechanisms 28; or otherwise modify the implant. In this regard, the detachable segments can be coupled to any suitable portion of the implant body, including a first lateral portion, a second lateral portion, an anterior portion, a posterior portion, a caudal portion, a cranial portion, a central portion, a medial portion, at a passage 26, proximate one or more fixation mechanisms 28, or any other suitable portion or combination of portions of the implant body. By way of non-limiting illustration, FIG. 1A shows some embodiments in which detachable segments 24 are coupled to lateral portions of the implant body. FIG. 3 shows some embodiments in which the implant 20 comprises one or more detachable segments 24 that are coupled to a lateral, anterior, or posterior portion of the implant body 22. FIG. 5A shows some embodiments in which one or more detachable segments 24 are coupled at a cranial portion of the implant body 22. FIG. 5B shows some embodiments in which one or more detachable segments 24 are coupled at a cranial portion and a caudal portion of the implant body 22. FIG. 6 shows some embodiments in which one or more detachable segments 24 are coupled at a cranial portion, lateral portions, and medial portions (proximate a passage 26) of the implant body 22. FIG. 7 shows some embodiments in which one or more detachable segments 24 are coupled (or couplable) at one or more lateral portions or posterior portions (and, in some embodiments, anterior portions) of the implant body 22 (e.g., with FIG. 7 showing some embodiments in which multiple detachable segments are coupled to both a lateral portion and posterior portion of the implant body 22). Indeed, FIG. 7 shows that, in some embodiments, one or more detachable segments (e.g., 24a or 24b) extend to an anterior portion of the implant 20 (e.g., from a posterior portion, to a lateral portion, or to an anterior portion).

Where the implant 20 comprises detachable segments 24, the implant can comprise any suitable number of segments, including 1, 2, 3, 4, 5, 6, 7, 8, 9, 10, or more. By way of non-limiting illustration, FIGS. 1A-1B and 2 show some embodiments in which 4 detachable segments 24 are coupled to the implant body 22. FIG. 5B shows some embodiments in which 6 detachable segments 24 are coupled to the implant body 22.

In some cases, detachable segments 24 couple directly to the implant body 22, and in some cases, detachable segments couple indirectly to the implant body by coupling to one or more other detachable segments or other components.

As mentioned, one or more detachable segments 24 are configured to be selectively removed to modify a size, footprint, contact area, or other aspect of the implant 20. To illustrate, FIG. 2 shows some embodiments of the implant 20 with a height H labeled, and FIG. 3 shows some embodiments of the implant 20 with a width W and a depth D labeled. In accordance with some embodiments, the detachable segments include any suitable segments configured to detach to thereby decrease the width, the depth, the height, or otherwise modify an angle or any other suitable measurement, shape, volume, or other characteristic of the implant. Accordingly, some embodiments are configured to go from a first measurement (e.g., one or more of a first width, a first depth, a first height, or any other suitable first measurement or shape) to a second measurement (e.g., one or more of a second width, a second depth, a second height, or any other suitable second measurement or shape) by removing one or more detachable segments. Indeed, in some embodiments, removing a single detachable segment 24 (e.g., as shown in FIG. 1C) is sufficient to transition from the first measurement or shape to the second measurement or shape, whereas in some embodiments, the implant is configured for multiple detachable segments to be removed in order to transition from the first measurement or shape to the second measurement or shape. For example, some embodiments include a first detachable segment on a first side (e.g., on a left lateral side) and a second detachable segment on a second side (e.g., on a right lateral side), wherein removal of both detachable segments keeps the implant symmetrical and results in the second width, depth, or height as desirable.

In some embodiments, one or more additional detachable segments 24 are provided to thereby allow for the implant 20 to transition from the second measurement to a third measurement or shape (with an even smaller width, depth, height, angle, shape, volume, or other measurement or characteristic), a fourth measurement or characteristic, or any additional number of measurements or characteristics (e.g., with increasingly smaller proportions). By way of non-limiting illustration, FIG. 1A shows an implant 20 with an implant body 22 having a first set of detachable segments 24a (wherein detachment of the first set of detachable segments takes the implant body 22 from a first width to a second width) and a second set of detachable segments 24b (wherein detachment of the second set of detachable segments takes the implant body from the second width to a third width).

The detachable segments 24 can be configured to transition the implant 20 from any suitable first measurement (or shape) to any suitable second measurement (or shape). For example, detachment of one or more detachable segments can be configured to decrease the size of the implant from the first measurement by between about 0.1 mm and 20 mm and any subrange thereof. For example, in some embodiments, detachment of one or more detachable segments decreases the size of the implant from the first measurement to the second measurement in an amount of about 0.1 mm, 0.15 mm, 0.2 mm, 0.25 mm, 0.3 mm, 0.5 mm, 0.75 mm, 1 mm, 1.5 mm, 2 mm, 2.5 mm, 3 mm, 4 mm, 5 mm, 6 mm, 7 mm, 8 mm, 9 mm, 10 mm, 11 mm, 12 mm, 15 mm, 17.5 mm, 20 mm, or any other suitable amount (in each case, optionally ± about 5%, 10%, 15%, 20%, 25%, 30%, 50%, or any other suitable amount). Indeed, some embodiments of the implant 20 are configured to have one or more width W measurements (e.g., the implant has a first width measurement when one or more detachable segments are included, and a second width measurement when one or more detachable segments are removed (and any suitable number of additional width measurements)), depth measurements, height measurements, or any other suitable measurements. For example, some embodiments are configured to decrease the measurement (e.g., the width, depth, height, or other measurement) by any suitable amount, such as one or more of the amounts listed in this paragraph.

As further examples, some embodiments have one or more depth D measurements (with inclusion or removal of one or more detachable segments 24) of about 22 mm, 23 mm, 24 mm, 25 mm, 26 mm, 27 mm, 28 mm, 29 mm, 30 mm, 31 mm, 32 mm, and any other suitable depth (as an example, some embodiments have a first depth of about 30 mm (or any other suitable first depth), then through removal of one or more detachable segments, the depth can be changed to 27 mm, 24 mm, or any other suitable second depth). By way of non-limiting illustration, FIG. 3 shows an implant 20 with one or more detachable segments 24 configured to change the depth D of the implant 20 from a first depth to a second depth via removal of the detachable segments 24.

Moreover, some embodiments are configured to have one or more width W measurements (with inclusion or removal of one or more detachable segments) of about 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, and any other suitable width (as an example, some embodiments have a first width of about 40 mm (or any other suitable first width), which can be decreased to 36 mm, 32 mm, or any other suitable second width through removal of one or more detachable segments). By way of non-limiting illustration, FIG. 3 shows an implant 20 with one or more detachable segments 24 configured to change the width W of the implant 20 from a first width to a second width via removal of the detachable segments 24.

Further, some embodiments are configured to have one or more height measurements (with inclusion or removal of one or more detachable segments) of about 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or any other suitable height (e.g., 10 mm, 12 mm, 14 mm, 16 mm, 18 mm, and 20 mm; or any other suitable combination). Thus, for example, an implant can have a first height of 18 mm (or any other suitable first height), and then through removal of one or more removable segments, transition to a second height of 16 mm (or any other suitable second height). By way of non-limiting illustration, FIG. 5B shows an implant 20 with one or more detachable segments 24 configured to change the height H of the implant 20 from a first height to a second height via removal of the detachable segments 24.

According to some embodiments, the implant 20 includes one or more lordotic, kyphotic, or other angles. In some embodiments, one or more detachable segments 24 are configured to alter the lordotic, kyphotic, or other angles (e.g., to provide a greater or lesser angle) of the implant or the implant body 22. By way of non-limiting illustration, FIGS. 5A-6 show some embodiments in which one or more detachable segments 24 are selectively removable to allow for an angle or shape of the implant 20 to be modified.

Some embodiments of the implant 20 provide one or more lordotic, kyphotic, or other angles of between 0 degrees and 35 degrees, or any other subrange thereof. For example, some embodiments include one or more lordotic or kyphotic angles of 0 degrees, 1 degree, 2 degrees, 3 degrees, 4 degrees, 5 degrees, 6 degrees, 7 degrees, 8 degrees, 9 degrees, 10 degrees, 11 degrees, 12 degrees, 13 degrees, 14 degrees, 15 degrees, 16 degrees, 17 degrees, 18 degrees, 19 degrees, 20 degrees, 21 degrees, 22 degrees, 23 degrees, 24 degrees, 25 degrees, 26 degrees, 27 degrees, 28 degrees, 29 degrees, 30 degrees, or any other suitable angle (e.g., 8 degrees, 14 degrees, 20 degrees, and 25 degrees; or any other suitable combination). Thus, for example, the implant can have an original angle of 0 degrees (or any other suitable first angle), then through removal of one or more detachable segments, the angle can be changed to 14 degrees (or any other suitable second angle). By way of non-limiting illustration, FIG. 5B shows an implant 20 with an original angle of approximately 0 degrees, wherein removal of one or more detachable segments 24 can change the angle.

The detachable segments 24 can have any suitable shape that allows them to function as described herein. Indeed, in some embodiments, one or more detachable segments comprise one or more linear objects, plates, bars, tabs, struts, rails, rods, slats, or other elongated objects. That said, the detachable segments can also have any other shape, such that a footprint or other shape or angle of the implant 20 has any suitable shape or size after separation of one or more detachable segments. For example, in some embodiments, the detachable segments include one or more wedges, curved objects, waved objects, crescents, contoured objects, arched objects, corrugated objects, polygonal objects, curvilinear polygonal shaped objects, claw-shaped objects, C-shaped objects, U-shaped objects, kidney-shaped objects, circular segment-like objects, elliptical segment-like objects, symmetrical objects, asymmetrical objects, or any other suitable shape that allows the size, shape, angle, footprint, volume, or other characteristic of the implant 20 to be modified.

By way of non-limiting illustration, FIG. 1A shows some embodiments in which a lateral-most detachable segment 24a comprises a segment of the implant 20 having a curved lateral-most edge and a relatively straight medial edge (comprising one or more interfaces 38). FIG. 1A also shows that, in some embodiments, a detachable segment, such as detachable segment 24b, is somewhat slab shaped, with the lateral edge of the segment running substantially parallel with the medial edge of the segment. FIGS. 5A and 5B show some embodiments in which one or more detachable segments comprise a wedge-shaped portion, a sloped portion, or a tapered portion. FIG. 6 shows some embodiments in which one or more detachable segments comprise a rounded portion (e.g., a rounded lateral edge of a lateral segment), a polygonal portion (e.g., a polygonal portion disposed adjacent to a passage 26), or a partially polygonal portion (e.g., part of a detachable segment disposed on the cranial (or upper) side of the implant body 22 shown in FIG. 6). Additionally, FIG. 7 shows some embodiments in which one or more detachable segments 24 have a crescent shape, a curved shape, or a claw-like shape (e.g., as shown by segments 24a and 24b in FIG. 7). FIG. 7 also shows some embodiments in which one or more detachable segments 24 have a bow-shape, a curved shape, or a curvilinear shape (e.g., as shown by segments 24c and 24d). Moreover, FIGS. 8A and 8B show some embodiments in which one or more detachable segments comprise a C-shaped object; a U-shaped object; a curved object; or a nested, internally tangent shape, including a nested, internally tangent family of homothetic shapes (e.g., as shown by segments 24a and 24b in FIGS. 8A and 8B).

The detachable segments 24 can include any of the features of the implant body 22. For example, in some embodiments, one or more of the detachable segments include an extension of the porous structure. Moreover, in some embodiments, the detachable segments are configured to have the same or similar strength, stiffness, pore size, or any other desirable feature of the implant body.

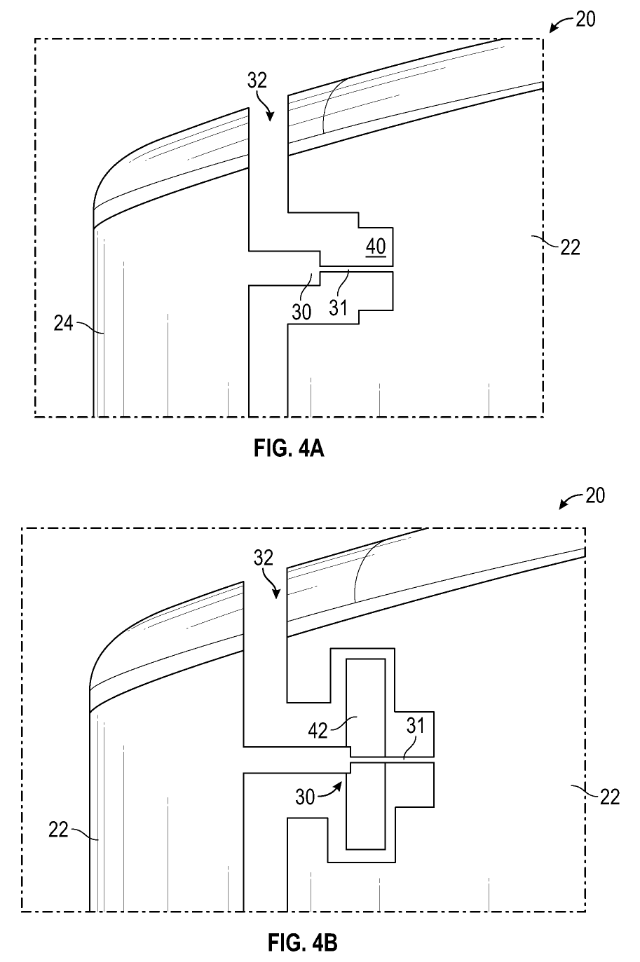

The detachable segments 24 can be configured to detach from (and, in some cases, reattach to) the implant body 22 in any suitable manner. For example, some embodiments of the detachable segments are selectively coupled to the implant body via one or more couplings 30, such as one or more breakaway couplings (e.g., a breakaway coupling having a breakaway portion); magnetic couplings; interference-fit couplings; friction-fit couplings; tongue-and-groove couplings; interlocking couplings; threaded fasteners or other anchors (e.g., nails, pins, shear pins, shear bolts, screws, bolts, staples, eyelets, snaps, buttons, zippers, ties, rivets, stakes, or any other anchors); hook-and-loop fasteners; snap-off tabs; adhesives; magnets; catches; mechanical engagements; or any other suitable couplings. By way of non-limiting illustration, FIG. 4A shows an implant body 22 with a detachable segment 24 coupled to the implant body via a coupling 30 in the form of a breakaway coupling.

The coupling 30 can couple between the implant body 22 and the corresponding detachable segment 24 in any suitable manner. Indeed, in some embodiments, the coupling couples to the implant body and the detachable segment (before the segment’s separation) such that a portion of the detachable segment abuts a portion of the implant body, contacts a portion of the implant body, is separated from a portion of the implant body by one or more gaps, or is disposed in any other suitable manner.

The detachable segments 24 can be positioned with respect to the implant body 22 or each other in any suitable manner. For example, they can be in contact with each other, separated from each other by one or more gaps 32, or otherwise disposed. Where the detachable segments and the implant body are configured to contact one another, any suitable portion of an inner face of a detachable segment can contact any suitable portion of an outer face of the implant body, including between about 1% and 100% of the respective faces, or any subrange thereof, such as about 25%, 50%, 75%, or any other suitable amount of contact. Where a gap 32 is included, the gap can have any suitable width (e.g., between 1 µm and 5 mm, or any subrange thereof).

Additionally, the coupling 30 and the detachable segment 24 can have any suitable configuration to allow the implant 20 to function properly with a larger footprint, with a different shape, with a different angle, or with another different characteristic when compared to the footprint, shape, angle, or other characteristic of the implant when the detachable segment is detached. For example, in some embodiments, the coupling is flexible to allow for the implant to conform to a greater degree to the spinal endplates without separation of the detachable segment. According to some embodiments, the coupling is disposed within an interior of the implant as opposed to at its surface. That said, in some embodiments, the coupling is (additionally or alternatively) disposed at the surface (which may, in some cases, be particularly useful in connection with stiffer implants).

The detachable segments 24 can be configured to detach from the implant body 20 in any suitable manner, including by breaking away in one or more particular manners, sliding in one or more particular directions, twisting away, magnetically separating, or otherwise detaching in any other suitable manner. In some embodiments, the interface between the implant body and the detachable segments is configured to prevent overloading of attached detachable segments 24 except in one or more particular directions. For example, in some cases, breaking of a breakaway coupling or other detachment of a detachable segment is configured to occur only when the detachable segment is forced in a particular direction with respect to the implant body 22. Indeed, in some cases, the detachable segment is configured to be moved directly apart from the implant body (e.g., within approximately 0 degrees to 45 degrees of directly opposite-facing vectors, or within any subrange thereof, such as within 2 degrees, 5 degrees, 10 degrees, 15 degrees, or any other suitable angle with respect to opposite-facing vectors) in order to detach. In some embodiments, separation occurs only through substantial shear forces (e.g., moving the implant body up (cranially), while moving the detachable segment down (caudally), or moving the implant body back (dorsally or posteriorly) while moving the detachable segment forward (ventrally or proximally), or, in either case, vice versa). In some cases, the detachable segment is configured to separate from the implant body when a combination of motions is applied to the detachable segment or implant body (e.g., a shear force plus a separation force).

According to some embodiments, the detachable segments 24 are configured to remain attached when experiencing any forces normally experienced during normal physiological implant use. Thus, in some cases, the detachable segments only detach when intentional actions are taken to detach them.

According to some embodiments, the detachable segments 24 are configured to selectively detach without the need for use of a particular instrument. For example, in some embodiments, pulling, twisting, sliding, or otherwise acting upon the detachable segments by hand is sufficient to detach them. That said, in some cases, the detachable segments are configured to detach only through use of a tool 34, or to more easily detach when a tool is used.

Where the detachable segments 24 are configured to be detached with the aid of a tool 34, any suitable tool can be used. For example, the tool may include one or more blades, pliers, forceps, scissors, spreaders, rods, shafts, drivers, screw drivers, levers, cams, or any other tools suitable for use in detaching the detachable segments. In some embodiments, the tool has one or more tips 36 configured to interface with the implant 20. By way of non-limiting illustration, FIG. 1B shows a tool 34 having a tip 36 configured to interface with an implant 20 to detach one or more detachable segments 24.

Although the tool 34 can interface with the implant 20 in any suitable manner, some embodiments of the implant include one or more interfaces 38 configured to receive or otherwise interface with the tool (or a part thereof, such as the tip 36). Although the interface can include any suitable interface for interacting with the tool, some embodiments of the interface include one or more access ports that allow access to the coupling 30 (or part thereof), that allow the tool to be wedged between and to pry apart the implant body and a detachable segment, or that otherwise allow the tool to selectively detach a detachable segment from the implant body. By way of non-limiting illustration, FIGS. 3 and 5B show some embodiments of implants 20 having a number of interfaces 38 configured to receive a tool (such as the tool 34 of FIG. 1B) to thereby detach one or more detachable segments 24.

According to some embodiments, the tool 34 is configured to be applied (e.g., to the interface 38) and operated in a manner that creates a force in a loading direction that is not substantially experienced by the implant 20 in vivo. For example, in some cases (e.g., in which the implant is configured to be exposed to significant caudally and cranially directed forces, as are typically encountered by an interbody spacer between spinal endplates) the tool is configured to apply a medial-lateral shear force (although some embodiments of the tool are configured to apply forces in any other suitable direction or directions).

The coupling 30 can be positioned between the applicable detachable segment 24 and the implant body 22 (or between multiple detachable segments) in any suitable manner. For example, according to some embodiments, the coupling 30 is positioned within (or extends through) a gap 32 between the implant body 22 and the applicable detachable segment 24 (or between multiple detachable segments).

Where the coupling 30 includes a breakaway coupling, the breakaway coupling can be configured in any suitable manner. In some embodiments, one or more particular portions of the breakaway coupling (breakaway portions 31) are configured to selectively break. For example, in some cases, the breakaway portion is thinner than one or more surrounding portions. In some embodiments, the breakaway portion is made from a more fragile or brittle material. In some embodiments, the breakaway portion is scored, perforated, weakened, configured to react to a stimulus (e.g., heat, light, pH, or another stimulus), configured to be broken (e.g., cut or otherwise broken) by the tool, or otherwise configured to break before other components surrounding the breakaway portion break. By way of non-limiting illustration, FIGS. 4A and 4B show some embodiments of couplings 30 in the form of breakaway couplings having a breakaway portion 31.

While the breakaway portion of the coupling 30 can have any suitable relation to one or more of the implant body 22 and the detachable segment 24, in some embodiments, the breakaway portion is (fully or partially) “sheltered” or disposed within a recess 40 such that one or more broken edges (which can, in some cases, be sharper than unbroken components once the detachable segment is separated) are not exposed. Indeed, in some cases, the breakaway portion 31 (or a part of it that is left when the detachable segment is separated from the implant body) is configured to be entirely housed within the implant body or a detachable segment (e.g., within a recess). By way of non-limiting illustration, FIG. 4A shows some embodiments in which a coupling 30 that includes a breakaway portion 31 is disposed within a recess 40 defined by the implant body 22.

According to some embodiments, the coupling 30 includes one or more locks 42. The lock can include any component configured to selectively prevent the detachable segment 24 from being detached (partially or completely). Indeed, in some embodiments in which the lock is engaged, the coupling cannot be separated. In contrast, in some embodiments in which the lock is disengaged, the coupling can be separated so the detachable segment can be detached. By way of non-limiting illustration, FIG. 4B shows a coupling 30 with a lock 42, wherein the lock prevents the coupling from being decoupled while the lock is engaged.

In some embodiments, the lock 42 is configured to be selectively engaged or disengaged using the tool 34. In some cases, the lock is housed within the recess 40 to help prevent inadvertent disengagement.

Although the lock 42 can be configured to function in any suitable manner (e.g., using one or more cylindrical locking mechanisms, pin-in-hole locking mechanisms, pins, screws, threaded engagements, dovetails, slots, grooves, rails, detents, frictional engagements, mechanical engagements, or any other suitable locking mechanisms), some embodiments are configured to prevent movement of the breakaway portion (or a detachable segment) in a breaking direction (or so as to completely separate from the implant body) while the lock is engaged. In some embodiments, the lock is configured to be disengaged via removal (or movement in a certain manner) of the lock.

In some cases, removal of the lock 42 occurs in a direction that is different from the breaking direction of the breakaway portion 31. Some embodiments of the lock are configured to twist away, be released with a key, be removed from a socket, translate with respect to a portion of the implant body 22, or to otherwise be manipulated to allow decoupling of the coupling 30 and to allow separation of a detachable segment 24 from the implant body.

By way of non-limiting illustration, FIG. 4B shows some embodiments in which a coupling 30 includes a breakaway coupling with a breakaway portion 31 configured to break when the detachable segment 24 is pulled apart from the implant body 22, but in which a lock 42 is present that selectively prevents undesired separation of the detachable segment 24 from the implant. However, in accordance with some embodiments, when the breakaway portion is broken, the lock can be disengaged in any suitable manner (e.g., by removing it, rotating it, dissolving it, forcing it through an egress, translating it with respect to a portion of the implant body, or otherwise displacing it) so that the lock no longer prevents separation of the detachable segment from the implant body. Indeed, FIG. 4B shows that, in some embodiments, when the breakaway portion 31 is broken, the detachable segment 24 can only be separated from the implant body 22 by being translated with respect to the implant body 22 (e.g., through an anterior-to-posterior translation). As a result, even during accidental breakage of the breakaway portion, the detachable segment would remain intact with the implant body when the implant 20 is implanted into a patient’s body such that anterior-to-posterior translation would be difficult (if not impossible) to obtain due to surrounding tissue. Indeed, in some embodiments, the implant comprises one or more lips, catches, detents, flaps, or other obstructions that selectively prevent the lock (and hence the detachable segment) from unintentionally sliding out of (or translating with respect to) the implant body, or vice versa, unless and until a sufficient force in one or more specific directions is applied.

According to some embodiments, the implant 20 (e.g., the implant body 22 or one or more detachable segments 24) includes one or more fixation mechanisms 28. The fixation mechanism can include any component suitable for coupling the implant to a bone (e.g., a spinal vertebra or another bone) or any other suitable portion of a patient. For example, some embodiments of the fixation mechanism include one or more anchors, one or more anchor receptacles, or any other suitable components. Where the fixation mechanism includes one or more anchors, the anchor can include any suitable anchor for coupling the implant to a bone or other relevant portion of a patient. For example, the anchor can include any suitable screw, blade, pin, bolt, hook, wire, rod, barbed anchors, flanged posts, or other anchor. The anchor can be configured to be at least partially disposed in any suitable anchor passage, or otherwise coupled to the implant in any suitable manner. For instance, the fixation mechanism can include any fixation mechanism or component thereof as described in U.S. Patent Application No. 18/623,983 (Attorney Docket No. 23845.179), entitled Stand-Alone Interbody Fusion, filed Apr. 1, 2024, or U.S. Patent Application No. 19/046,436 (Attorney Docket No. 23845.211), filed Feb. 5, 2025, entitled Systems and Methods for Providing and Deploying Interbody Implants, the entire disclosures of each of which are incorporated herein by this reference. By way of non-limiting illustration, FIGS. 1A-3 show some embodiments in which the fixation mechanism 28 includes one or more receptacles 29 that are configured to receive one or more anchors (not shown).

According to some embodiments, the fixation mechanism 28 is configured to receive anchors of different sizes. For example, where the fixation mechanism is configured to receive screws, some embodiments are configured to receive screws of different lengths. For example, some embodiments are configured to selectively receive one or more screws having lengths of anywhere between 5 mm and 50 mm (e.g., screws having lengths of about 20 mm, 25 mm, 30 mm, and 35 mm (in each case, optionally ± 1%, 3%, 5%, 10%, or any other suitable amount)). Some embodiments are configured to receive screws having different diameters. Some embodiments can receive screws having diameters of anywhere between 1.5 mm and 7.5 mm. For example, some embodiments can receive both screws having diameters of 5 mm and screws having diameters of 5.5 mm (in each case, optionally ± 1%, 3%, 5%, 10%, or any other suitable amount).

The fixation mechanism 28 can be configured to receive anchors of different sizes in any suitable manner. For example, some embodiments include multiple anchor receptacles, with different receptacles being configured to receive anchors of different sizes. In some embodiments, one or more anchor receptacles are adjustable. For instance, in some cases, one or more receptacles include one or more detachable segments 24, wherein detachment of such detachable segments results in a receptacle configured to receive a different size anchor. By way of non-limiting illustration, FIG. 3 shows an implant 20 having detachable segments 24 and a fixation mechanism 28, wherein the fixation mechanism 28 is configured to receive a first size of anchor (not shown) when certain of the detachable segments are attached (in particular, the ventral, or front, detachable segments), and a second size of anchor when one or more of such detachable segments are detached.

The described systems and components can be made in any suitable manner, including via molding, extrusion, etching, casting, machining, CNC milling, micro milling, milling, drilling, electrical discharge machining, forging, stamping, rolling, plasma spraying, thermal oxidation, laser surface melting, electroplating, micro-arc oxidation, nanostructuring, additive manufacturing, or in any other suitable manner. Indeed, according to some embodiments, one or more portions of the implant 22 are formed using additive manufacturing. For example, in some cases, the coupling 30 is formed via additive manufacturing.

The described implant 20 and any portion thereof can be made from or otherwise comprise any suitable material. In some embodiments, one or more portions of the implant include one or more of PEEK, stainless steel, tantalum, zirconium, a niobium alloy, titanium, a titanium alloy, a cobalt-nickel-chromium-molybdenum alloy, porous titanium, CoCr, a polymer, plastic, a ceramic, or any other suitable implant material. In some embodiments, the coupling 30 is modified to ensure appropriate functionality based on the type of material used. For example, some embodiments that use a stronger material have a coupling with a thinner breakaway portion 31, or a breakaway portion formed of a different material.

The described systems and methods can be modified in any suitable manner. For instance, in some embodiments, multiple detachable segments 24 are provided on a cranial or caudal surface (e.g., as shown in FIG. 6), wherein removal of a first detachable segment (e.g., 24a or 24b) changes the angle, height, shape, footprint, volume, or other characteristic of the implant 20, and removal of both the first 24a and the second 24b detachable segments changes the overall height, angle, or other characteristic of the implant 20.

As another example of a suitable modification, in some embodiments, one or more detachable segments 24 are configured to reattach (in the same place or a different place on the implant body 22). For example, in some cases, a lateral portion can be detached and added to a cranial portion, thereby both decreasing the footprint and increasing the height of the implant 20.

In accordance with some embodiments, any other suitable detachment and reattachment is also allowed. For instance, in some cases, two or more detachable segments 24 are interchangeable (e.g., to provide different shape, size, angle, or contour features). For example, in some embodiments, a first detachable segment can be removed and replaced with a second detachable segment that provides a different size, shape, contour, stiffness, angle, or other implant feature.

As yet another example of a suitable modification, in some embodiments, one or more detachable segments 24 are configured to be removed or added to the implant body 22 via decoupling of one or more screws or other anchors. While this can be accomplished in any suitable manner, in some embodiments, at least a part of the fixation mechanism 28 (or one or more secondary fixation mechanisms) is included on one or more of the detachable segments, wherein in some cases, the detachable segments are free of the fixation mechanism or part thereof.

As still another example of a suitable modification, while some embodiments of the implant 20 are configured to remain relatively symmetrical when the detachable segments 24 are detached (e.g., because a detachable segment is removed from both lateral sides of the implant), in some embodiments, one or more detachable segments can be removed to leave the implant body with an asymmetrical shape.

In addition to the aforementioned features, the described systems and methods can include any other suitable feature. Indeed, in some cases, a hospital’s or practitioner’s implant inventory size can be greatly reduced using the systems and methods described herein, while still providing a full range of implants (in some cases, an even broader range than would be found with different implants for every combination of desirable features). Moreover, in some embodiments, instead of trying to use an implant that is not specifically tailored for a particular patient, the described systems and methods allow practitioners to specifically tailor an implant for each particular patient.

As another example of a suitable feature, some embodiments of the described implant 20 are readily customizable to one or more sizes that are not generally provided by some competing manufacturers. Indeed, in some cases, a medical practitioner may discover during surgery, when the access provided by the practitioner or other surgeon is limited in width, that an implant needs to be relatively narrow and deep. In such cases, the practitioner typically would like to maximize the footprint of the implant to provide the most stability and the most osteoconductive surface. Indeed, being that the surgical access is narrow to a disc space that is deep, the practitioner typically wants to intraoperatively match the implant to the space. In this regard, some embodiments of the described implant can readily be customized (e.g., via removal of one or more detachable segments 24) to tailor and match the implant to the patient’s specific needs and anatomy.

As another example, unlike some competing implant devices that can be cut or milled down to size (e.g., requiring a saw or other abrasive tool), some embodiments of the described implant 20 are prepared with discrete and specific portions (e.g., detachable segments 24) that are configured to be selectively removed (e.g., with a lever or in any other suitable manner).

Any and all of the components in the figures, embodiments, implementations, instances, cases, methods, applications, iterations, elements, and other parts of this disclosure can be combined and mixed and matched in any suitable manner. Additionally, any component can be removed, separated from other components, modified with or without modification of like components, or otherwise altered together or separately from anything else disclosed herein.

As used herein, the singular forms “a,” “an,” “the,” and other singular references include plural referents, and plural references include the singular, unless the context clearly dictates otherwise. For example, reference to a fixation mechanism 28 includes reference to one or more fixation mechanisms, and reference to detachable segments 24 includes reference to one or more detachable segments. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including,” “having,” “such as,” “for example,” “e.g.,” and any similar terms are not intended to limit the disclosure, and may be interpreted as being followed by the words “without limitation.”

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

1. An interbody implant comprising:
an implant body; and
a first detachable segment selectively coupled to the implant body, wherein the interbody implant has a first size when the first detachable segment is coupled to the implant body, and a second size when the first detachable segment is detached from the implant body.

2. The interbody implant of claim 1, wherein the first detachable segment is coupled to a lateral portion of the implant body.

3. The interbody implant of claim 1, wherein the first detachable segment is coupled to at least one of a ventral portion and a dorsal portion of the implant body.

4. The interbody implant of claim 1, wherein the first detachable segment is coupled to at least one of a cranial portion and a caudal portion of the implant body.

5. The interbody implant of claim 1, further comprising a second detachable segment.

6. The interbody implant of claim 5, wherein the second detachable segment is coupled to the first detachable segment.

7. The interbody implant of claim 5, wherein the second detachable segment is disposed across the implant body from the first detachable segment, substantially opposite the first detachable segment.

8. The interbody implant of claim 5, wherein the first detachable segment is coupled to at least one of a first lateral portion of the implant body, a second lateral portion of the implant body, a ventral portion of the implant body, a dorsal portion of the implant body, a cranial portion of the implant body, and a caudal portion of the implant body, and wherein the second detachable segment is coupled to a different one of the first lateral portion of the implant body, the second lateral portion of the implant body, the ventral portion of the implant body, the dorsal portion of the implant body, the cranial portion of the implant body, and the caudal portion of the implant body.

9. The interbody implant of claim 1, wherein the first detachable segment is coupled to the implant body via a coupling comprising a breakaway portion.

10. The interbody implant of claim 9, wherein the breakaway portion is disposed within a recess in at least one of the implant body and the first detachable segment.

11. The interbody implant of claim 1, further comprising a second detachable segment and a third detachable segment.

12. An interbody implant comprising:
a cranial surface;
a caudal surface;
an implant body disposed between the cranial surface and the caudal surface; and
a detachable segment selectively coupled to the implant body via a coupling, wherein detachment of the detachable segment from the implant body modifies a characteristic of the interbody implant.

13. The interbody implant of claim 12, further comprising an interface whereby a tool can be used to separate the detachable segment from the implant body.

14. The interbody implant of claim 12, further comprising a gap between the implant body and the detachable segment.

15. The interbody implant of claim 12, wherein at least a portion of the coupling is disposed within a recess in at least one of the implant body and the detachable segment.

16. The interbody implant of claim 12, further comprising a lock configured to prevent the detachment of the detachable segment from the implant body when the lock is engaged.

17. The interbody implant of claim 12, wherein the coupling comprises a breakaway coupling having a breakaway portion.

18. The interbody implant of claim 12, wherein the characteristic comprises a size.

19. The interbody implant of claim 12, wherein the characteristic comprises an angle.

20. A method of modifying an interbody implant, the method comprising:
detaching a detachable segment of the interbody implant from an implant body of the interbody implant to thereby modify a characteristic of the interbody implant.
